(19) United States
(12) Patent Application Publication
(10) Pub. No.: US 2023/0098296 A1
(43) Pub. Date: Mar. 30, 2023

(54) METHOD AND SYSTEM FOR GENERATING DATA SET RELATING TO FACIAL EXPRESSIONS, AND NON-TRANSITORY COMPUTER-READABLE RECORDING MEDIUM

(71) Applicant: UX FACTORY CO.,LTD., Seongnam-si (KR)

(72) Inventors: Min Young AHN, Yongin-si (KR); Jun Young PARK, Seoul (KR); Chung Heorn LEE, Suwon-si (KR); Yu Min SEO, Seoul (KR)

(73) Assignee: UX FACTORY CO.,LTD., Seongnam-si (KR)

(21) Appl. No.: 18/077,442

(22) Filed: Dec. 8, 2022

Related U.S. Application Data
(63) Continuation of application No. PCT/KR2021/008070, filed on Jun. 28, 2021.

(30) Foreign Application Priority Data
Jul. 7, 2020 (KR) 10-2020-0083755

(51) Int. Cl. A61B 5/16; A61B 5/00; G06F 16/55; G06F 16/51

(52) U.S. Cl. CPC A61B 5/16; A61B 5/0077; G06F 16/55; G06F 16/51; G06V 40/174

(57) ABSTRACT

A method for generating a data set relating to facial expressions is provided. The method includes the steps of: acquiring information on a user's facial expressions specified while the user takes a psychological test, and information on an analysis result of the psychological test associated with the information on the user's facial expressions; labeling the information on the user's facial expressions with reference to the information on the analysis result of the psychological test; and generating a data set containing the information on the user's facial expressions and information on the labeling.

### CROSS-REFERENCE TO RELATED APPLICATION(S)

This application is a continuation application of Patent Cooperation Treaty (PCT) International Application No. PCT/KR2021/008070 filed on Jun. 28, 2021, which claims priority to Korean Patent Application No. 10-2020-0083755 filed on Jul. 7, 2020. The entire contents of PCT International Application No. PCT/KR2021/008070 and Korean Patent Application No. 10-2020-0083755 are hereby incorporated by reference.

### FIELD OF THE INVENTION

The present invention relates to a method, system, and non-transitory computer-readable recording medium for generating a data set relating to facial expressions.

### BACKGROUND

Facial expressions are one of communication methods for conveying human emotions and intentions, and various studies on facial expression recognition are being conducted to understand human emotions. In particular, in recent years, many techniques have been developed that can accurately recognize changes in facial expressions and classify emotions.

However, according to the techniques introduced so far, data on facial expressions are collected in a state where a user intentionally makes specific expressions according to instructions of a collection manager in order to increase the accuracy of classifying facial expressions and emotions, so that the data are inevitably intentional and biased, and utilizing the data has a negative impact on the accuracy of analyzing facial expressions. Although it is possible to consider utilizing facial expression data sets proposed by American scientist Paul Ekman, the data mainly relate to white males and thus it is difficult to extensively apply the data to other races or genders.

Many attempts have been made to acquire data on natural facial expressions and concurrent emotions. However, even if the natural facial expressions are acquired, it is often ambiguous to classify the facial expressions or concurrent emotions, which causes a problem that accurate labeling is difficult.

In this connection, the inventor(s) present a novel and inventive technique capable of generating an accurate data set relating to facial expressions by associating a psychological test result with facial expression data.

### SUMMARY OF THE INVENTION

One object of the present invention is to solve all the above-described problems in prior art.

Another object of the invention is to enable accurate labeling by associating information on facial expressions acquired during a psychological test with information on an analysis result of the psychological test.

Yet another object of the invention is to generate an accurate and highly useful data set relating to facial expressions.

The representative configurations of the invention to achieve the above objects are described below.

According to one aspect of the invention, there is provided a method for generating a data set relating to facial expressions, comprising the steps of: acquiring information on a user's facial expressions specified while the user takes a psychological test, and information on an analysis result of the psychological test associated with the information on the user's facial expressions; labeling the information on the user's facial expressions with reference to the information on the analysis result of the psychological test; and generating a data set containing the information on the user's facial expressions and information on the labeling.

According to another aspect of the invention, there is provided a system for generating a data set relating to facial expressions, comprising: an information acquisition unit configured to acquire information on a user's facial expressions specified while the user takes a psychological test, and information on an analysis result of the psychological test associated with the information on the user's facial expressions; a labeling management unit configured to label the information on the user's facial expressions with reference to the information on the analysis result of the psychological test; and a data set generation management unit configured to generate a data set containing the information on the user's facial expressions and information on the labeling.

In addition, there are further provided other methods and systems to implement the invention, as well as non-transitory computer-readable recording media having stored thereon computer programs for executing the methods.

According to the invention, it is possible to enable accurate labeling by associating information on facial expressions acquired during a psychological test with information on an analysis result of the psychological test.

According to the invention, it is possible to generate an accurate and highly useful data set relating to facial expressions.

### BRIEF DESCRIPTION OF THE DRAWINGS

FIG. 1 schematically shows the configuration of an entire system for generating a data set relating to facial expressions according to one embodiment of the invention.

FIG. 2 illustratively shows the internal configuration of a management system according to one embodiment of the invention.

FIG. 3 illustratively shows how to generate a data set relating to facial expressions and perform learning on the basis of the data set according to one embodiment of the invention.

FIG. 4 illustratively shows how to generate a data set relating to facial expressions and perform learning on the basis of the data set according to one embodiment of the invention.

### DETAILED DESCRIPTION

In the following detailed description of the present invention, references are made to the accompanying drawings that show, by way of illustration, specific embodiments in which the invention may be practiced. These embodiments are described in sufficient detail to enable those skilled in the art to practice the invention. It is to be understood that the various embodiments of the invention, although different from each other, are not necessarily mutually exclusive. For example, specific shapes, structures, and characteristics described herein may be implemented as modified from one embodiment to another without departing from the spirit and scope of the invention. Furthermore, it shall be understood that the positions or arrangements of individual elements within each embodiment may also be modified without departing from the spirit and scope of the invention. Therefore, the following detailed description is not to be taken in a limiting sense, and the scope of the invention is to be taken as encompassing the scope of the appended claims and all equivalents thereof. In the drawings, like reference numerals refer to the same or similar elements throughout the several views.

Hereinafter, various preferred embodiments of the present invention will be described in detail with reference to the accompanying drawings to enable those skilled in the art to easily implement the invention.

Configuration of the Entire System

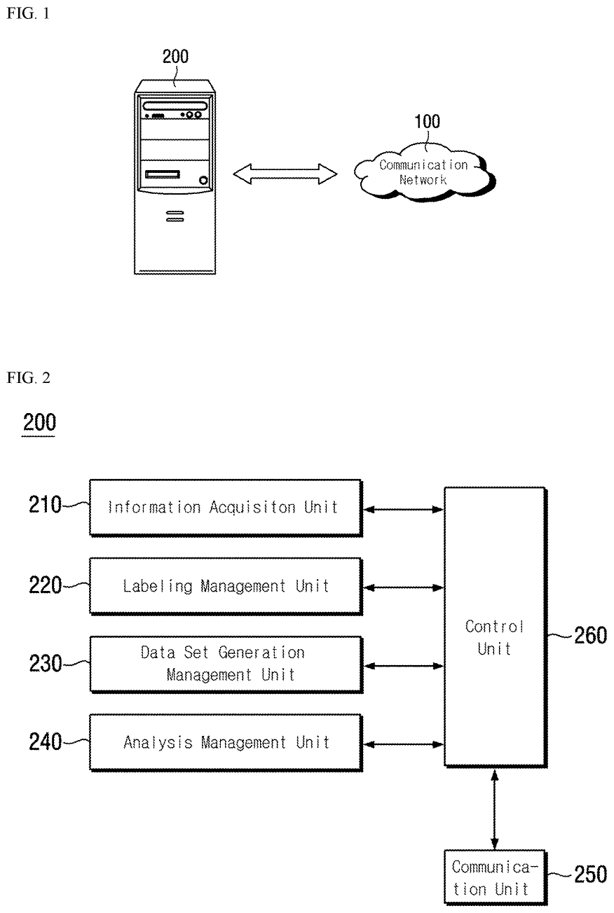

FIG. 1 schematically shows the configuration of the entire system for collecting data relating to facial expressions according to one embodiment of the invention.

As shown in FIG. 1, the entire system according to one embodiment of the invention may comprise a communication network 100 and a management system 200.

First, the communication network 100 according to one embodiment of the invention may be implemented regardless of communication modality such as wired and wireless communications, and may be constructed from a variety of communication networks such as local area networks (LANs), metropolitan area networks (MANs), and wide area networks (WANs). Preferably, the communication network 100 described herein may be the Internet or the World Wide Web (WWW). However, the communication network 100 is not necessarily limited thereto, and may at least partially include known wired/wireless data communication networks, known telephone networks, or known wired/wireless television communication networks.

For example, the communication network 100 may be a wireless data communication network, at least a part of which may be implemented with a conventional communication scheme such as WiFi communication, WiFi-Direct communication, Long Term Evolution (LTE) communication, Bluetooth communication (more specifically, Bluetooth Low Energy (BLE) communication), infrared communication, and ultrasonic communication. As another example, the communication network 100 may be an optical communication network, at least a part of which may be implemented with a conventional communication scheme such as LiFi (Light Fidelity).

Next, the management system 200 according to one embodiment of the invention may function to: acquire information on a user's facial expressions specified while the user takes a psychological test, and information on an analysis result of the psychological test associated with the information on the user's facial expressions; label the information on the user's facial expressions with reference to the information on the analysis result of the psychological test; and generate a data set containing the information on the user's facial expressions and information on the labeling.

The functions of the management system 200 will be discussed in more detail below. Meanwhile, the above description is illustrative although the management system 200 has been described as above, and it will be apparent to those skilled in the art that at least a part of the functions or components required for the management system 200 may be implemented in another management system 200 or included in an external system (not shown), as necessary.

Configuration of the Management System

Hereinafter, the internal configuration of the management system 200 crucial for implementing the invention and the functions of the respective components thereof will be discussed.

FIG. 2 illustratively shows the internal configuration of the management system 200 according to one embodiment of the invention.

As shown in FIG. 2, the management system 200 according to one embodiment of the invention may comprise an information acquisition unit 210, a labeling management unit 220, a data set generation management unit 230, an analysis management unit 240, a communication unit 250, and a control unit 260. According to one embodiment of the invention, at least some of the information acquisition unit 210, the labeling management unit 220, the data set generation management unit 230, the analysis management unit 240, the communication unit 250, and the control unit 260 may be program modules to communicate with an external system (not shown). The program modules may be included in the management system 200 in the form of operating systems, application program modules, or other program modules, while they may be physically stored in a variety of commonly known storage devices. Further, the program modules may also be stored in a remote storage device that may communicate with the management system 200. Meanwhile, such program modules may include, but are not limited to, routines, subroutines, programs, objects, components, data structures, and the like for performing specific tasks or executing specific abstract data types as will be described below in accordance with the invention.

First, the information acquisition unit 210 according to one embodiment of the invention may function to acquire information on a user's facial expressions specified while the user takes a psychological test, and information on an analysis result of the psychological test associated with the information on the user's facial expressions. The psychological test according to one embodiment of the invention may include at least one question associated with the user's emotion (or disposition), and more specifically, may be a test for classifying or specifying the user's emotion (or disposition) on the basis of each question or a plurality of questions. Further, according to one embodiment of the invention, the information on the user's facial expressions may include information on a movement, change, pattern, metric, or feature specified on the basis of a predetermined region or landmark of the face to recognize the facial expressions, or information on a movement, change, pattern, metric, or feature specified with respect to a predetermined action unit of a facial body part (e.g., a muscle). In addition, the information on the analysis result of the psychological test according to one embodiment of the invention may include information on an emotion (or disposition) or a type thereof specified with reference to at least one question that the user answers while taking the psychological test, such as information on an emotion (or disposition) or a type thereof specified on the basis of a relationship between a plurality of questions that the user answers while taking the psychological test (or between answers to the plurality of questions).

For example, the information acquisition unit 210 may acquire the information on the user's facial expressions in time series while the user takes the psychological test. More specifically, the information acquisition unit 210 may acquire the information on the user's facial expressions by specifying the information on the user's facial expressions in time series while the user takes the psychological test, and representing the specified information in predetermined block units. Here, the block unit may refer to a unit specified on the basis of a predetermined expression unit (which may refer to, for example, each of a smiling expression and an angry expression when the smiling expression and the angry expression appear consecutively) or a predetermined question unit (i.e., at least one question unit associated with a specific emotion) (which may refer to, for example, three questions when the three questions are associated with a specific emotion).

Further, the information acquisition unit 210 may specify the information on the analysis result of the psychological test with reference to at least one of at least one expert comment associated with the psychological test and biometric information of the user specified while the user takes the psychological test. For example, the user's biometric information may include information on at least one of brain waves, pulse waves, heartbeats, body temperature, a blood sugar level, pupil changes, blood pressure, and an amount of oxygen dissolved in blood.

For example, the information acquisition unit 210 may use a result of at least one expert's emotion analysis (or disposition analysis) acquired on the basis of at least one question of the psychological test or the user's answer to the at least one question (i.e., an expert comment) and biometric information acquired while the user answers the question of the psychological test to supplement or verify the analysis result derived from a result of the answer to the question of the psychological test.

More specifically, when “happiness” is derived as the user's emotion from a result of an answer to a question of the psychological test whereas “annoyance” is specified as the user's emotion on the basis of the user's biometric information or a result of an expert's emotion analysis is contrary to “happiness”, the information acquisition unit 210 may specify the information on the analysis result of the psychological test by excluding the result of the answer to the question of the psychological test, or calculating, comparing, and analyzing scores on the basis of weights respectively assigned to the result of the answer to the question of the psychological test, the user's biometric information, and the result of the expert's emotion analysis.

Next, the labeling management unit 220 according to one embodiment of the invention may function to label the information on the user's facial expressions with reference to the information on the analysis result of the psychological test.

For example, the labeling management unit 220 may label the information on the user's facial expressions with reference to an emotion associated with at least one question of the psychological test. More specifically, the labeling management unit 220 may match an emotion specified in at least one question of the psychological test with information on the user's facial expression acquired while the user answers the at least one question. For example, when the emotion of “happiness” is specified in at least one question of the psychological test, information on the user's facial expression may be matched with information on “happiness” while the user answers the at least one question.

Next, the data set generation management unit 230 according to one embodiment of the invention may function to generate a data set containing the information on the user's facial expressions and information on the labeling.

For example, the data set generation management unit 230 may pack the information on the user's facial expressions and the information on the emotions labeled therefor (i.e., the information on the labeling) as a bundle (or as a unit set) to generate a data set containing a plurality of bundles.

Next, the analysis management unit 240 according to one embodiment of the invention may perform learning associated with facial expression analysis on the basis of the data set generated by the data set generation management unit 230. According to one embodiment of the invention, the learning associated with facial expression analysis may include a variety of learning related to face recognition, emotion recognition, and the like which may be performed on the basis of facial expression analysis. It is noted that the types of learning according to the invention are not necessarily limited to those listed above, and may be diversely changed as long as the objects of the invention may be achieved.

For example, the analysis management unit 240 according to one embodiment of the invention may acquire information on a feature, pattern, or metric of a facial expression corresponding to each of a plurality of emotions from the data set, and train a learning model using the information as learning data, thereby generating a learning model associated with facial expression analysis (e.g., a learning model capable of estimating an emotion of a person from a facial image of the person). The learning model may include a variety of machine learning models such as an artificial neural network or a deep learning model. For example, the learning model may include a support vector machine model, a hidden Markov model, a k-nearest neighbor model, and a random forest model.

Next, according to one embodiment of the invention, the communication unit 250 may function to enable data transmission/reception from/to the information acquisition unit 210, the labeling management unit 220, the data set generation management unit 230, and the analysis management unit 240.

Lastly, according to one embodiment of the invention, the control unit 260 may function to control data flow among the information acquisition unit 210, the labeling management unit 220, the data set generation management unit 230, the analysis management unit 240, and the communication unit 250. That is, the control unit 260 according to the invention may control data flow into/out of the management system 200 or data flow among the respective components of the management system 200, such that the information acquisition unit 210, the labeling management unit 220, the data set generation management unit 230, the analysis management unit 240, and the communication unit 250 may carry out their particular functions, respectively.

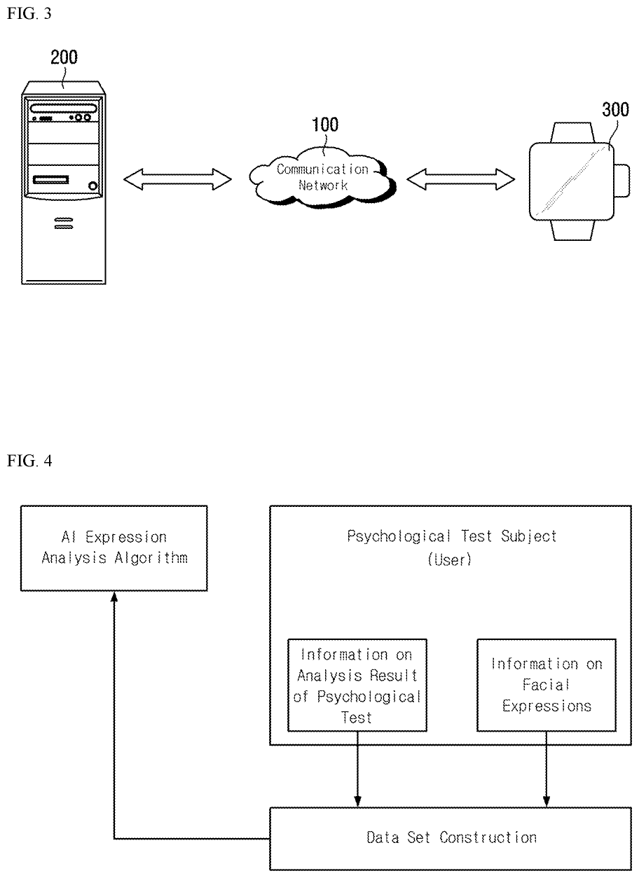

FIGS. 3 and 4 illustratively show how to collect data relating to facial expressions and perform learning on the basis of the data according to one embodiment of the invention.

Referring to FIG. 3, it may be assumed that the user wears a wearable device 300 and takes a psychological test.

The wearable device 300 according to one embodiment of the invention is digital equipment that may function to connect to and then communicate with the management system 200, and may be portable digital equipment, such as a smart watch or smart glasses, having a memory means and a microprocessor for computing capabilities.

Further, according to one embodiment of the invention, the functions of at least one of the information acquisition unit 210, the labeling management unit 220, and the analysis management unit 240 of the management system 200 may be performed in the wearable device 300, and the wearable device 300 according to one embodiment of the invention may provide the user with a user interface necessary to perform the above functions.

First, according to one embodiment of the invention, information on the user's facial expressions may be acquired in time series through the wearable device 300 while the user takes the psychological test.

Next, information on an emotion corresponding to at least one question of the psychological test is acquired with reference to a result of the user's answer to the at least one question, and a result of an expert's emotion analysis may be provided as an expert comment with respect to the result of the user's answer to the at least one question of the psychological test.

Next, information on an analysis result of the psychological test may be acquired with reference to the information on the emotion corresponding to the at least one question and the result of the expert's emotion analysis. Meanwhile, the information on the analysis result of the psychological test may be acquired with further reference to biometric information of the user acquired through the wearable device 300 while the user takes the psychological test.

Next, the information on the user's facial expressions may be labeled on the basis of the information on the analysis result of the psychological test.

Next, a data set containing the information on the user's facial expressions and information on the labeling may be generated.

Next, at least one learning model for emotion recognition based on facial expression analysis may be generated on the basis of the generated data set.

Next, an emotional state may be specified by analyzing the facial expressions on the basis of the at least one learning model.

The embodiments according to the invention as described above may be implemented in the form of program instructions that can be executed by various computer components, and may be stored on a computer-readable recording medium. The computer-readable recording medium may include program instructions, data files, and data structures, separately or in combination. The program instructions stored on the computer-readable recording medium may be specially designed and configured for the present invention, or may also be known and available to those skilled in the computer software field. Examples of the computer-readable recording medium include the following: magnetic media such as hard disks, floppy disks and magnetic tapes; optical media such as compact disk-read only memory (CD-ROM) and digital versatile disks (DVDs); magneto-optical media such as floptical disks; and hardware devices such as read-only memory (ROM), random access memory (RAM) and flash memory, which are specially configured to store and execute program instructions. Examples of the program instructions include not only machine language codes created by a compiler, but also high-level language codes that can be executed by a computer using an interpreter. The above hardware devices may be changed to one or more software modules to perform the processes of the present invention, and vice versa.

Although the present invention has been described above in terms of specific items such as detailed elements as well as the limited embodiments and the drawings, they are only provided to help more general understanding of the invention, and the present invention is not limited to the above embodiments. It will be appreciated by those skilled in the art to which the present invention pertains that various modifications and changes may be made from the above description.

Therefore, the spirit of the present invention shall not be limited to the above-described embodiments, and the entire scope of the appended claims and their equivalents will fall within the scope and spirit of the invention.

What is claimed is:

1. A method for generating a data set relating to facial expressions, comprising the steps of:
acquiring information on a user's facial expressions specified while the user takes a psychological test, and information on an analysis result of the psychological test associated with the information on the user's facial expressions;
labeling the information on the user's facial expressions with reference to the information on the analysis result of the psychological test; and
generating a data set containing the information on the user's facial expressions and information on the labeling.

2. The method of claim 1, wherein in the acquiring step, the information on the user's facial expressions is specified in time series while the user takes the psychological test.

3. The method of claim 2, wherein the information on the user's facial expressions is represented in predetermined block units.

4. The method of claim 1, wherein in the labeling step, the information on the user's facial expressions is labeled with reference to an emotion associated with at least one question of the psychological test.

5. The method of claim 1, wherein the information on the analysis result of the psychological test is specified with reference to at least one expert comment associated with the psychological test.

6. The method of claim 5, wherein the information on the analysis result of the psychological test is specified with reference to biometric information of the user specified while the user takes the psychological test.

7. The method of claim 1, further comprising the step of performing learning associated with facial expression analysis on the basis of the generated data set.

8. A non-transitory computer-readable recording medium having stored thereon a program for executing the method of claim 1.

9. A system for generating a data set relating to facial expressions, comprising:
an information acquisition unit configured to acquire information on a user's facial expressions specified while the user takes a psychological test, and information on an analysis result of the psychological test associated with the information on the user's facial expressions;
a labeling management unit configured to label the information on the user's facial expressions with reference to the information on the analysis result of the psychological test; and
a data set generation management unit configured to generate a data set containing the information on the user's facial expressions and information on the labeling.

10. The system of claim 9, wherein the information acquisition unit is configured to specify the information on the user's facial expressions in time series while the user takes the psychological test.

11. The system of claim 10, wherein the information on the user's facial expressions is represented in predetermined block units.

12. The system of claim 9, wherein the labeling management unit is configured to label the information on the user's facial expressions with reference to an emotion associated with at least one question of the psychological test.

13. The system of claim 9, wherein the information on the analysis result of the psychological test is specified with reference to at least one expert comment associated with the psychological test.

14. The system of claim 13, wherein the information on the analysis result of the psychological test is specified with reference to biometric information of the user specified while the user takes the psychological test.

15. The system of claim 9, further comprising an analysis management unit configured to perform learning associated with facial expression analysis on the basis of the generated data set.
